(19) United States
(12) Patent Application Publication
(10) Pub. No.: US 2025/0025387 A1
(43) Pub. Date: Jan. 23, 2025

(54) COMPOSITION FOR THE TREATMENT OF THE SKIN OF DIABETES PATIENTS

(71) Applicant: MIBELLE AG, Buchs (CH)

(72) Inventors: Fred ZÜLLI, Küttigen (CH); Franz SUTER, Döttigen (CH); Franziska WANDREY, Zürich (CH); Cornelia SCHÜRCH, Staufen (CH)

(73) Assignee: MIBELLE AG, Buchs (CH)

(21) Appl. No.: 18/714,942

(22) PCT Filed: Nov. 22, 2022
(86) PCT No.: PCT/EP2022/082692

§ 371 Date: May 30, 2024

(30) Foreign Application Priority Data
Dec. 2, 2021 (EP) 21211909.3

(51) Int. Cl. A61K 8/19; A61K 8/14; A61K 8/55; A61Q 17/00

(52) U.S. Cl. CPC A61K 8/19; A61K 8/14; A61K 8/553; A61Q 17/00

(57) ABSTRACT

Pharmaceutical or cosmetic composition having an active content of divalent calcium for use in topical treatment of skin with impaired barrier function, especially preferably the skin of diabetes patients, wherein the divalent calcium is present in the composition as a phospholipid complex.

### TECHNICAL FIELD

The present invention relates to compositions for treatment of the skin of diabetes patients or prediabetes patients, in particular for prevention of dry skin or for restoration of a sufficiently hydrated skin and/or for restoration of the barrier effect and/or for prevention of wrinkling. Furthermore, the composition preferably leads to a reduction of itching, redness, blistering, furunculosis and/or tightness. The present invention further relates to therapeutic or cosmetic uses of such compositions and to methods of preparing such compositions.

### PRIOR ART

Skin changes occur in the vast majority of people with diabetes, these occurring not only at a late stage of the disease, but as early as at the prediabetic stage with elevated blood sugar levels. Pathological skin changes are promoted by poor metabolic management. Skin disorders such as, in particular, dry skin, associated severe itching, blistering, redness, wrinkles or furuncles can be caused by a poorly managed diabetes disease, but even with good management, it is frequently not possible to prevent, in particular, dry skin. Diabetics should therefore use special products for care of their sensitive skin. Even if metabolism is well managed, people with diabetes are more readily susceptible to fungal and other skin infections, and about a third suffer from skin dysfunction because of an excessively high blood sugar level. Diabetic skin resembles aged skin. In particular, the barrier function of the skin is impaired.

Cosmetic or medical products which result in good skin hydration are available for care of diabetic dry skin. However, these products only control the symptoms of diabetic skin, but not the cause thereof.

Good skin hydration is achieved by using occlusive lipids such as shea butter, lanolin, mineral oils and a multitude of other fats. Furthermore, these products use high concentrations of moisturizing factors, such as urea. As a result of all these components, it is not very pleasant to handle these care products. They are greasy and are easily removed on clothing.

Devaz and Pal in “Abstracts for 6th Central European Symposium on Pharmaceutical Technology and Biotechnology ED—Zakelj Simon; Mrhar Ales; Gra”, EUROPEAN JOURNAL OF PHARMACEUTICAL SCIENCES, ELSEVIER AMSTERDAM, NL, vol. 25, 1 May 2005, P-27, page S83-page S84, describe the preparation of cochleates as phospholipid-calcium precipitates having a molar ratio of calcium to lecithin in the range of 1:2. Therapeutic applications are not specified.

Hirotsuka et al. in “Calcium fortification of soy milk with calcium-lecithin liposome system”, JOURNAL OF FOOD SCIENCE, WILEY-BLACKWELL PUBLISHING, INC, US, vol. 49, no. 4, describe how calcium ions can be provided in the form of lecithin liposomes; therapeutic applications are not specified here either.

### DISCLOSURE OF THE INVENTION

Accordingly, it is inter alia an object of the present invention to propose pharmaceutical or cosmetic compositions for topical application, i.e., in particular as a cream or ointment, which provide relief or even improvement in skin properties for people with skin with impaired barrier function, and/or for diabetes patients or prediabetes patients, in particular precisely those with skin with impaired barrier function.

A skin with impaired barrier function is to be understood herein to mean a skin exhibiting increased transepidermal water loss (TEWL). This variable is measured, for example, using a Tewameter® TM300 instrument, Courage+Khazaka, Germany. A skin with impaired barrier function is accordingly typically to be understood to mean a skin with a TEWL value greater than 12 g/m²/h, preferably greater than 15 g/m²/h, especially preferably greater than 20 g/m²/h or even greater than 25 g/m²/h. As is customary, the value is measured on the inside of the forearm.

In other words, a skin with impaired barrier function is typically to be understood to mean a skin with a TEWL value at least 4 g/m²/h, preferably at least 7 g/m²/h, especially preferably at least 15 g/m²/h or even at least 20 g/m²/h above the normal value (assuming measurement of the normal value on the inside of the forearm again. Healthy adult subjects typically have an average TEWL value of 6.8 g/m²/h, again measured on the inside of the forearm (cf. for example Akdeniz, M., Gabriel, S., Lichterfeld-Kottner, A., Blume-Peytavi, U. and Kottner, J. (2018), TEWL reference values in healthy adults. Br J Dermatol, 179: e204-e204. https://doi.org/10.1111/bjd.17215).

Such pharmaceutical or cosmetic compositions for topical application are thus proposed in particular for prevention of dry skin or for restoration of a sufficiently hydrated skin and/or for restoration of the barrier effect and/or for prevention of wrinkling. Furthermore, the composition preferably leads to a reduction of itching, redness, blistering, furunculosis and/or tightness. In particular, it is intended that, inter alia, the cause of dry skin and not just its symptoms be controlled. Furthermore, it is also intended that the convenience of application be improved.

It is intended that the restoration of the skin barrier and hence skin hydration be achieved especially preferably without occlusive fats and urea.

Accordingly, the proposed formulations, as semifinished product but in particular the dosage form thereof intended for use, for example as an ointment, cream, lotion, paste or tincture for local application to the skin of the patient, are preferably free of urea (or contain less than 1% by weight or less than 0.5% by weight or less than 0.25% by weight thereof), and/or they are formulated in such a way that they do not form a closed layer in the form of a film on the skin (nonocclusive). The proposed formulations are therefore especially preferably also free of (or contain less than 1% by weight or less than 0.5% by weight or less than 0.25% by weight of) one of the following substances or a combination thereof: siloxanes, 10 Vaseline, lanolins, mineral oils, and/or other occlusive substances, in particular selected from the following group: petrolatum, C18-C30 alkyl methyl siloxanes; dimethicones; polymethylsilsesquioxanes; lanolin or lanolin alcohol; mineral oil (paraffinum liquidum); preference being given to these substances, in combination or individually, being absent or present in less than 1% by weight or less than 0.5% by weight or less than 0.25% by weight.

The present invention accordingly provides a pharmaceutical and/or cosmetic composition as claimed in claim 1 or uses of such compositions and methods for therapeutic and/or cosmetic treatment, and also methods for preparing such compositions.

According to a first aspect, the invention relates to a pharmaceutical or cosmetic composition having an active content of divalent calcium for use in topical treatment of the skin of diabetes patients or prediabetes patients, wherein the divalent calcium is present in the composition as a phospholipid complex.

Preferably, the phospholipid complex is in the form of an aqueous gel in such a composition and/or in the starting material comprising calcium phospholipid for the composition. A gel is to be understood in line with the IUPAC Gold Book (https://doi.org/10.1351/goldbook.G02600) according to general knowledge in this field to mean a nonfluid colloidal network or polymer network that is expanded through its whole volume by a fluid. A gel has a finite, usually rather small, yield stress. A gel can contain a covalent polymer network, for example a network formed by crosslinking polymer chains or by nonlinear polymerization. A gel can also contain a polymer network formed through the physical aggregation of polymer chains, caused by hydrogen bonds, crystallization, helix formation, complexation, etc, that results in regions of local order acting as the network junction points. The resulting swollen network may be termed a thermoreversible gel if the regions of local order are thermally reversible. A gel can also contain a polymer network formed through glassy junction points, for example one based on block copolymers. If the junction points are thermally reversible glassy domains, the resulting swollen network may also be termed a thermoreversible gel.

It has been found that the formulation of the divalent calcium as a phospholipid complex, in particular a special phospholipid complex with a double-conical structure, unexpectedly results in the availability on topical application, for example as a concentrate but also as a cream or ointment, being substantially higher than if calcium is added, for example, as a simple chloride or similar salt to such a formulation. In particular, the skin barrier functionally provided by an intact stratum corneum is rebuilt.

The phospholipids present in the proposed complex exhibit no effect here when topically applied alone, for example as a phospholipid mixture, as will be explained below. It is the formulation comprising phospholipid complex in specific combination with calcium that rebuilds the skin barrier.

As will be explained below, the formulation of the divalent calcium in this form can stabilize, reduce or even reverse skin problems typical of diabetes patients. These include in particular the treatment of skin problems due to diabetes or prediabetes, in particular dry skin or moisturization of dry skin, atopic skin conditions, the restoration of the barrier function of the skin, the stabilization, prevention, reduction or elimination of skin infections caused by lack of barrier function, in particular of bacterial origin, or fungal infections, pigment disorders (diabetic dermatopathy), blistering, itching (pruritus diabeticorum), redness (including necrobiosis lipoidica diabeticorum, pseudoacanthosis nigricans, bullosis diabeticorum, rubeosis diabeticorum, scleroedema diabeticorum), wrinkling, scaling skin, tightness, wound healing disorders, elasticity.

Preferably, the proposed formulation is used for dermatological treatment of diabetes patients, preferably type 2 patients.

The proposed composition is preferably characterized in that the phospholipid is a phosphoglyceride (lecithin) selected from the following group: phosphatidic acid; phosphatidylcholine; phosphatidylethanolamine; phosphatidylinositol; phosphatidylserine; diphosphatidylglycerol (cardiolipin), or a mixture of these systems. These systems can be fully or partially hydrogenated or can be nonhydrogenated.

It is particularly preferred that the calcium phospholipid complex in the composition is at least partially a calcium complex with negatively charged phospholipids (in particular phosphatidic acid, phosphatidylinositol, phosphatidylserine and others).

This is preferably with a molar ratio of divalent calcium to phospholipid (based on the total amount of phospholipids, i.e., negatively charged and uncharged phospholipids) in the range of 0.05:1-20:1 (i.e., 0.05-20), preferably in the range of 0.1:1-5:1 (i.e., 0.1-5), especially in the range of 0.2:1-1:1 (i.e., 0.2-1), preferably in the range of 0.3:1-0.8:1 (i.e., 0.3-0.8).

In particular, the system is characterized in that the calcium phospholipid complex is in the form of a double-conical structure as preferably an at least partially neutral complex and is thus readily bioavailable (according to FIG. 1b). These structures are formed when phospholipids are suitably mixed together with Ca²⁺ solutions, and a cigar-shaped structure is formed that stabilizes the Ca²⁺ double cones in an aqueous environment (FIG. 1a). The complex is neutral especially when the Ca²⁺ is complexed with negatively charged phospholipids. However, since the phospholipids can complex the Ca²⁺ not only with negative charges, but also as an uncharged phospholipid with its partial charges, it is possible that, in the composition, the complex is entirely or partially formed with uncharged or charged phospholipid or is partly formed with uncharged phospholipid and partly formed with charged phospholipid.

Therefore, according to a further preferred embodiment, the calcium phospholipid complex is a calcium complex with phospholipids, in particular at least partially with phosphatidic acid, phosphatidylinositol, and/or phosphatidylserine, with a molar ratio of divalent calcium to negatively charged phospholipids, preferably phosphatidic acid, phosphatidylinositol, and/or phosphatidylserine, based on the content of negatively charged phospholipids, in the range of 0.1:1-30:1 (i.e., 0.1-30), preferably in the range of 0.3:1-10:1 (i.e., 0.3-10), more preferably in the range of 0.4:1-5:1 (i.e., 0.4-5) or 0.5:1-1:1 (i.e., 0.5-1).

The Ca²⁺ phospholipid complex can be prepared by using customary lecithins from soy, egg, sunflower and other sources. These lecithins differ in phospholipid composition. There are different head groups and different fatty acid compositions. Furthermore, these phospholipids in the lecithins can be treated yet further in order to achieve new properties, for example the hydrogenation of the fatty acids to increase their stability or the separation of the head group or the separation of the fatty acids from the glycerol structure to make the phospholipids smaller. Pure phospholipids from synthesis or purification can also be used besides natural sources. Furthermore, phospholipid-analogue molecules bearing a negatively charged polar head group and having a hydrophobic tail can also be used. These molecules can be found in the fields of emulsifiers and wash-active substances.

In the composition, for the proposed applications, the concentration of the divalent calcium in the Ca²⁺ phospholipid complex intended for topical application is typically in the range of 0.001-10 percent by weight, preferably in the range of 0.005-5.0 or 0.01-0.5 percent by weight (percent by weight of the CaCl₂·2H₂O based on the entire composition). In the composition, for the proposed application, the concentration of the phospholipids in the Ca²⁺ phospholipid complex intended for topical application is typically in the range of 0.01-30 percent by weight, preferably in the range of 0.05-5 percent by weight (percent by weight of the phospholipids based on the entire composition).

The proposed composition can be formulated as an ointment, cream, lotion, paste or tincture for local application to the skin of the patient.

The composition then preferably additionally contains at least one of the following formulation constituents: thickeners, smoothing agents, moisturizers and/or humectants, surface-active agents, preservatives, antifoams, waxes, fats, oils, antioxidants or substances with antioxidant properties, bactericides, fungicides, perfumes, propellants, dyes, stabilizers, polar and apolar solvents, in particular water, pigments, UV filters, plant extracts, further active therapeutic or pharmaceutical ingredients, or a combination thereof.

The composition can also be in the form a concentrate, for example as bulk material for the preparation of the formulations for end use, and be designed for the preparation of a topical application formulation with a concentration of the Ca²⁺ phospholipid complex in the composition in the range of 0.1-100 percent by weight, preferably in the range of 1-10 percent by weight (percent by weight of the Ca²⁺ phospholipid complexes based on the topical composition).

The present invention further relates to the use of such a composition for therapeutic and/or cosmetic treatment of the skin of a diabetes patient or to a method for therapeutic and/or cosmetic treatment of the skin of a diabetes patient. In particular, this concerns the stabilization, prevention, reduction or elimination of dry skin or moisturization of dry skin and/or the restoration of the impaired barrier function of the skin. Alternatively or additionally, this concerns the stabilization, prevention, reduction or elimination of skin infections caused by damaged barrier function, by skin changes or skin irritation, in particular of bacterial origin, or concerns the stabilization, prevention, reduction or elimination of fungal infections, pigment disorders (e.g., diabetic dermopathy), blistering, itching (e.g., pruritus diabeticorum), redness (including necrobiosis lipoidica diabeticorum, pseudoacanthosis nigricans, bullosis diabeticorum, rubeosis diabeticorum, scleredema diabeticorum), wrinkling, or else furunclosis and/or tightness, wherein a composition as described above is topically applied to the skin.

The composition is preferably used in formulated form as an ointment, cream, lotion, paste or tincture for local application to the skin of the patient.

The present invention further relates to a method for preparing a composition as described above, characterized in that the phospholipid starting material, optionally after prior dissolution or dispersion in an organic solvent, in particular ethanol, or a mixture of an organic solvent and water, is provided in an aqueous medium as a liposome structure, preferably having an average particle size of less than 300 nm and preferably less than 200 nm.

This dispersion is then admixed with an aqueous solution of Ca²⁺, wherein this aqueous solution has been preferably set at a pH in the range of 7.5-10 and especially preferably in the range of 8-9.

Either the dispersion containing liposomes can be added to the aqueous calcium solution, or the dispersion containing liposomes can be initially charged and the calcium solution added. This is preferably done while stirring. At the end of this process, the Ca²⁺ is in the form of double cones with suitable setting of the concentrations and temperatures and of the ratios between phospholipid and calcium.

Preferably, these steps are followed by homogenization to form a gel, thus forming the described aggregates.

Preferably, the preparation of the liposomes and/or the homogenization proceeds with the aid of a high-pressure homogenizer, preferably at a pressure of at least 500 bar and especially preferably at least 1000 bar.

An ointment, cream, lotion, paste or tincture can then be formulated from the concentrated composition with the aid of at least one carrier material and optionally further constituents. The Ca²⁺ phospholipid complexes (in particular double cones or aggregates thereof) can, in other words, be prepared as follows:

The phospholipid molecules, for example the lecithins, are transferred into a water phase which can additionally contain further components such as: ethanol, glycerol, panthenol, propylene glycol, further active ingredients such as moisturizers and active antiaging ingredients, preservatives, dyes, odorants, stabilizers, and other cosmetic/medical ingredients. The phospholipids can be transferred into the water phase by swelling or by prior dissolution in a solvent such as ethanol. Preference is given to preparing a liposome structure of the phospholipids (lecithin) in the water phase. Known methods for preparing liposome can be used, such as high-pressure homogenization, extrusion, dialysis, ultrasound and further methods.

Then, while stirring, a Ca²⁺ solution (e.g., obtained by dissolving calcium chloride CaCl₂·2H₂O) is added slowly to the phospholipid (lecithin) phase. Different Ca²⁺ salts can be used. In this mixture, the correct concentrations must be chosen so that the desired complex structures can form, in particular the double-cone Ca²⁺ structures (FIG. 1b). The appropriate proportions depend on the phospholipids (lecithins) used and can be tested in individual cases. Depending on the total concentration of the phospholipids, the formation of the Ca²⁺ phospholipid double-cone complexes leads to a gelatinous consistency, in particular for transfer into aggregates (FIG. 1a).

Further embodiments are specified in the dependent claims.

### BRIEF DESCRIPTION OF THE DRAWINGS

Preferred embodiments of the invention are described below on the basis of the drawings, which serve merely for elucidation and are not to be interpreted as limiting. In the drawings:

FIG. 1 shows complex phospholipid-Ca²⁺ structures, with a) showing an aggregate of double-cone structures and b) showing the underlying or individually occurring double-cone structure;

FIG. 2 shows hematoxylin-eosin stains of human 3D epidermis treated with different calcium concentrations or Ca²⁺ phospholipid complexes after 9 days of differentiation, with a) showing the control (1.1 mM CaCl₂·2H₂O basal), b) showing a reduced calcium concentration (0.3 mM CaCl₂·2H₂O basal), c) showing treatment with CaCl₂·2H₂O (0.3 mM CaCl₂·2H₂O basal and 1.1 mM CaCl₂·2H₂O apical), and d) showing treatment with Ca²⁺ phospholipid complex (0.3 mM CaCl₂·2H₂O basal and 0.1% Ca²⁺ phospholipid complex, prepared analogously to sample 6 in Table 1, apical); impairment of epidermis formation was observed with reduced calcium concentration (b) and with apical treatment with a 1.1 mM calcium chloride dihydrate solution (c); treatment with Ca²⁺ phospholipid complexes (d) supports the formation of a normal epidermis;

FIG. 3 shows loricrin expression stains of 3D epidermis treated with different calcium concentrations or Ca²⁺ phospholipid complexes after 9 days of differentiation, with a) showing the control (1.1 mM CaCl₂·2H₂O basal), b) showing a reduced calcium concentration (0.3 mM CaCl₂·2H₂O basal), c) showing treatment with CaCl₂·2H₂O (0.3 mM CaCl₂·2H₂O basal and 1.1 mM CaCl₂·2H₂O apical), and d) showing treatment with Ca²⁺ phospholipid complex (0.3 mM CaCl₂·2H₂O basal and 0.1% Ca²⁺ phospholipid complex, prepared analogously to sample 6 in Table 1, apical); reduced loricrin production was observed with reduced calcium concentration (b) and with treatment with a 1.1 mM calcium chloride dihydrate solution (c); treatment with Ca²⁺ phospholipid complexes (d) normalized the expression level of loricrin;

FIG. 4 shows hematoxylin-eosin stains of human 3D epidermis treated with different calcium concentrations, phospholipid mixtures or Ca²⁺ phospholipid complexes after 9 days of differentiation, with a) showing the control (1.1 mM CaCl₂·2H₂O basal), b) showing a reduced calcium concentration (0.3 mM CaCl₂·2H₂O basal), c) showing treatment with 0.1% phospholipid mixture (apical without Ca²⁺), and d) showing treatment with Ca²⁺ phospholipid complex (0.3 mM CaCl₂·2H₂O basal and 0.1% Ca²⁺ phospholipid complex, prepared analogously to sample 6 in Table 1, apical); impairment of epidermis formation was observed with reduced calcium concentration (b) and with apical treatment with 0.1% phospholipid mixture (without Ca²⁺) (c); treatment with Ca²⁺ phospholipid complexes (d) supports the formation of a normal epidermis; e) showing quantification of the epidermis thickness of a)-d), with specification of the measured epidermis thickness in um on the ordinate. ***p<0.001 versus untreated; **p<0.05 versus untreated; ####p<0.0001 versus Ca²⁺ reduced; ###p<0.001 versus phospholipid mixture (no Ca²⁺).

FIG. 5 shows a) involucrin stains of skin explants for determination of increase in involucrin expression, treated with placebo (top, apical), with a 0.013% calcium chloride dihydrate solution (bottom, 0.013% CaCl₂·2H₂O apical) or with Ca²⁺ phospholipid complexes also containing 0.013% calcium chloride dihydrate (middle, 2% Ca²⁺ phospholipid complex containing 0.013% CaCl₂·2H₂O, prepared analogously to sample 6 in Table 1, apical); b) quantification of involucrin expression of a), with specification of involucrin protein expression compared to the placebo (=100) in % on the ordinate;

FIG. 6 shows the change in various skin parameters after seven days of treatment with placebo gel (black bars) or 2% Ca²⁺ phospholipid complexes (hatched), followed in each case by a 24-hour application of 2% sodium lauryl sulfate (to impair the skin barrier), compared to the starting value, with specification of the change in skin parameters compared to untreated control in % on the ordinate and where: *P<0.05 versus untreated: **p<0.05 versus untreated and placebo; ***p<0.01 versus untreated and placebo.

### DESCRIPTION OF PREFERRED EMBODIMENTS

### Formation of a Ca²⁺ Phospholipid Complex Structure

A soy lecithin (containing 30-40% negatively charged phospholipids) was dissolved in alcohol and then transferred into the water phase and subjected to high-pressure homogenization, thereby producing liposomes having a diameter of about 100 nm. CaCl₂·2H₂O solutions of differing concentration were added to this liposome dispersion while stirring, and mixture was then mixed once again by means of a high-pressure homogenizer. The resultant products were examined for the formation of stable gel structures and described in Table 1 below. For the molar ratio values specified in Table 1, it was assumed that the phospholipids in the soy lecithin used have an average molar mass of 762 g/mol. The specified molar ratio is the ratio of Ca²⁺ to total lecithin (charged and uncharged). For the ratio of Ca²⁺ to negatively charged lecithin, the molar ratios in the table should be multiplied by 3, assuming that approx. 33% of the phospholipids in the lecithin are negatively charged.

TABLE 1
Various ratios for complex formation

| | Lecithin | | Molar ratio | Description |
|---|---|---|---|---|
| Sample | % | CaCl₂•2H₂O % | Ca²⁺:lecithin | of structure |
| 1 | 5 | 0 | nd | Monodisperse |
| | | | | transparent |
| | | | | liposomes |
| 2 | 5 | 0.04 | 0.041 | Relatively large |
| | | | | liposomes, |
| | | | | milky appearance |
| 3 | 5 | 0.08 | 0.083 | Unstable dispersion |
| | | | | with immediate phase |
| | | | | separation |
| 4 | 5 | 0.16 | 0.167 | Unstable dispersion |
| | | | | with phase |
| | | | | separation after |
| | | | | a few days |
| 5 | 5 | 0.32 | 0.333 | Stable milky gel |
| | | | | with high viscosity; |
| | | | | stable for months |
| 6 | 5 | 0.64 | 0.667 | Stable milky gel |
| | | | | with high viscosity; |
| | | | | stable for months |
| 7 | 5 | 1.28 | 1.333 | Immediate phase |
| | | | | separation |

nd: not determined

It is found in this experiment that stable Ca²⁺ phospholipid complexes (samples 5 and 6) are formed at suitable mixing ratios, for these lecithins here at molar ratios (total lecithin) in the range of 0.2:1.0-1:1 (i.e., 0.2-1.0), preferably in the range of 0.3:1-0.8:1 (i.e., 0.3-0.8), or at molar ratios (negatively charged lecithin) in the range of 0.6:1-3:1 (i.e., 0.6-3), preferably in the range of 1:1-2:1 (i.e., 1-2).

### Example 1

1.2 kg of glycerin, 0.6 kg of ethanol and 3.56 kg of ultrapure water are mixed at 60° C. This is followed by dispersion therein of 0.3 kg of soy lecithin having a content of 50% phosphatidylcholine. After 2 hours, the dispersion is pumped through a high-pressure homogenizer at 1200 bar. This operation is repeated 2 to 5 times until the average liposome particle size (Z average) is below 200 nm and the pH is 3-8.

In a second step, 0.3 kg of ultrapure water are initially charged, and 0.0378 kg of CaCl₂·2H₂O are dissolved therein and a pH of 8.5-9.5 set.

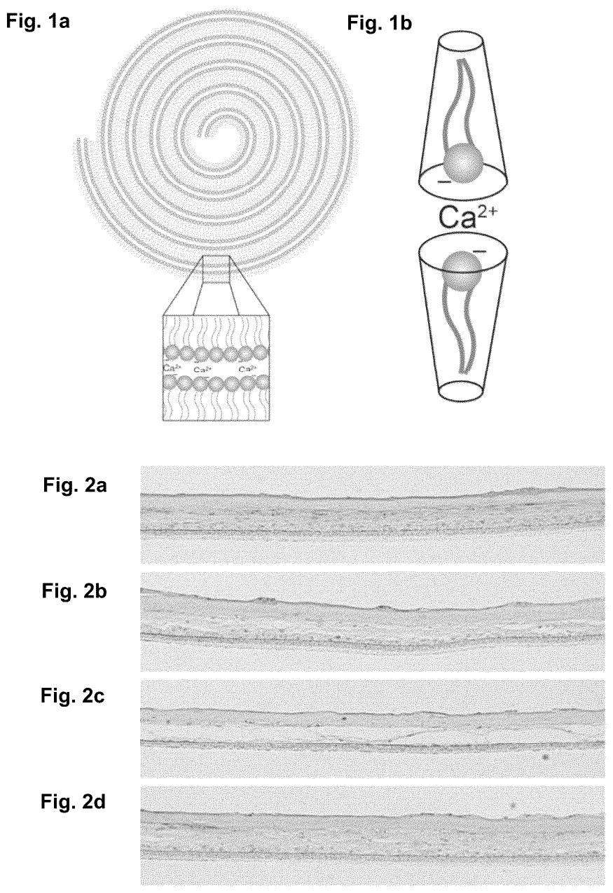

This CaCl₂ solution is then added slowly to the liposome dispersion while stirring. This forms a Ca²⁺ lecithin complex having an increased viscosity (double cones, FIG. 1b). To achieve a homogeneous structure, the entire complex is pumped through the high-pressure homogenizer once again at 1200 bar. The final Ca²⁺ complex having a gelatinous structure can then be filled into tubs and is provided for use in topical formulations (aggregated double cones, FIG. 1a).

The molar ratio of divalent calcium to lecithin is thus 0.667 at the end, and the example corresponds to sample 6 in Table 1.

### Example 2

1.2 kg of ethanol are used to dissolve 0.3 kg of soy lecithin having a content of 65% phosphatidylcholine at 60° C. This is followed by adding the solution to 4.16 kg of ultrapure water while stirring. This mixture is then pumped 3 times through a high-pressure homogenizer at 1200 bar, and the result is a liposome dispersion having an average particle size of 50 nm and a pH of 3-8.

In a second step, 0.3 kg of ultrapure water are initially charged, and 0.026 kg of CaCl₂·2H₂O are dissolved therein and a pH of 8.5 set.

This CaCl₂ solution is then added slowly to the liposome dispersion while stirring. This forms a Ca²⁺ phospholipid complex having an increased viscosity (double cones). This complex is then homogenized again at 1200 bar to produce a homogeneous gel structure which can then be used in creams, gels and other topical products (aggregated double cones). The molar ratio of divalent calcium to lecithin is thus 0.458 at the end.

### Preparation of Further Phospholipid Complexes Which can be Used in Combination with the Ca²⁺ Complexes

The method described can also be used for other divalent ions. Thus Zn²⁺, Cu²⁺, Mg²⁺, Mn²⁺, Sn²⁺, Mn²⁺, Fe²⁺ or other cations can also be incorporated into phospholipid complexes using similar methods. This can increase their biological activities and/or improve bioavailability.

### Example 3 (Zn²⁺)

100 g of glycerin, 50 g of pentylene glycol and 270 g of ultrapure water are mixed and heated to 50° C. This is followed by adding 25 g of lecithin from sunflower having a content of 50% phosphatidylcholine. Once the lecithin is well dispersed, the dispersion is homogenized twice at 1200 bar. The resultant liposomes have an average particle size of 150 nm. A second water phase then contains 2.92 g of ZnCl₂ in 12.73 g of water having a pH of 8.5. The second water phase is then added slowly to the liposome dispersion while stirring. This results in a gelatinous structure having a Zn²⁺ phospholipid complex.

### Application of the Ca²⁺ Phospholipid Complexes (Double Cones)

The Ca²⁺ phospholipid complex thus prepared (double cone aggregates), optionally in combination with other further complexes, can be applied directly to the skin in concentrated form in the form of the gelatinous structure or can be incorporated into cosmetic or medical formulations. Since the isolated Ca²⁺ double cone structure is not stabilized in water, the double-cone Ca²⁺ phospholipid complex takes a multiple structure and in the form of aggregated double cones having a macroscopically gelatinous structure (FIG. 1a). These structures can then be formulated into aqueous systems such as oil-in-water emulsions or purely aqueous systems.

### Formulation of the Ca²⁺ Phospholipid Complex in Various Cosmetic Products

TABLE 2
Formulation example 1; rich cream for dry skin

| | % by |
|---|---|
| INCI name | weight |
| Aqua | ad 100 |
| Glycerin | 2 |
| Sodium PCA | 2 |
| Sodium Lactate Solution | 2 |
| Pentylene Glycol | 7 |
| Xanthan Gum | 0.2 |
| Lactic Acid | 0.5 |
| Gluconolactone (and) Sodium Benzoate | 1 |
| Mangifera Indica (Mango) Seed Butter | 5 |
| Butyrospermum Parkii Butter | 5 |
| Shorea Stenoptera Butter | 2 |
| Prunus Amygdalus Dulcis (Sweet Almond) Oil | 10 |
| Coco-Caprylate/Caprate | 4 |
| Cetearyl Alcohol (and) Cetearyl Glucoside | 5 |
| Panthenol | 15 |
| Ca²⁺ phospholipid complex (double cone aggregates)* | 5 |
| Tocopherol | 0.2 |
| Perfume | q.s. |
| Sodium Hydroxide | q.s. |

*Complex according to example 1 above

TABLE 3
Formulation example 2; body lotion
containing Ca²⁺ phospholipid complex

| | % by |
|---|---|
| INCI name | weight |
| Aqua | ad 100 |
| Glyceryl Stearate (and) PEG-30 Stearate | 3 |
| Palmitic (and) Stearic Acid | 2 |
| Stearyl Alcohol | 1.2 |
| Cetyl Alcohol | 1.2 |
| Ca²⁺ phospholipid complex (double cone aggregates)* | 3 |
| 2-Phenoxyethanol | 1 |
| Sodium Hydroxide | q.s. |

*Complex according to example 1 above

TABLE 4
Formulation example 3; Ca²⁺ phospholipid complex gel

| | % by |
|---|---|
| INCI name | weight |
| Aqua | ad 100 |
| Acrylates/C10-30 Alkyl Acrylate Crosspolymer | 0.5 |
| Ca²⁺ phospholipid complex (double cone aggregates)* | 3 |
| 2-Phenoxyethanol | 1 |
| Sodium Hydroxide | q.s. |

*Complex according to example 1 above

### Activity of the Ca²⁺ Phospholipid Complexes

### Rebuilding of the Epidermis Structure in an In Vitro Skin Model, Control: CaCl₂

The effect of the Ca²⁺ phospholipid complexes on the differentiation of 3D-reconstructed epidermis was investigated. To this end, keratinocyte progenitor cells were cultivated in low-calcium growth medium (TAK-GM, 0.03 mM calcium chloride dihydrate (CaCl₂·2H₂O)) and were then used to reconstruct a 3D epidermis. For this purpose, the medium was replaced by a 3D differentiation medium (TAK-3D) containing 1.1 mM CaCl₂·2H₂O and the cells were grown in the medium for a further day. The cultures were then contacted with the air in order to induce differentiation and formation of the skin barrier, and grown for a further 9 days with changing of the medium every second day in the following way:
Condition 1: 1.1 mM CaCl₂·2H₂O from the basal side (TAK-3D). This is the standard condition for efficient epidermis formation.
Condition 2: 0.3 mM CaCl₂·2H₂O from the basal side (low calcium concentration). The calcium concentration was reduced here in order to cause impaired formation of the epidermis and thus produce an impaired skin barrier. This is the minimum necessary calcium concentration that must be present on the basal side in order to ensure formation of the epidermis.
Condition 3: 0.3 mM CaCl₂·2H₂O from the basal side+1.1 mM CaCl₂·2H₂O from the apical side. Here, calcium was additionally added to the 3D epidermis apically in the form of calcium chloride dihydrate. Apical administration was carried out for comparability with condition 4.
Condition 4: 0.3 mM CaCl₂·2H₂O from the basal side+0.1% Ca²⁺ phospholipid complexes (prepared analogously to sample 6 in Table 1) from the apical side in order to imitate topical application of the phospholipid complex. Basal treatment would be equivalent to systemic treatment with calcium.

The 3D models were removed on day 9 and processed for histological analysis. The hematoxylin-eosin stains was examined for stratification thereof under a microscope, and the epidermal differentiation marker loricrin was stained immunohistochemically (Biolegend, catalog number 905104). High-resolution micrographs (10× magnification) were taken (cf. FIG. 2).

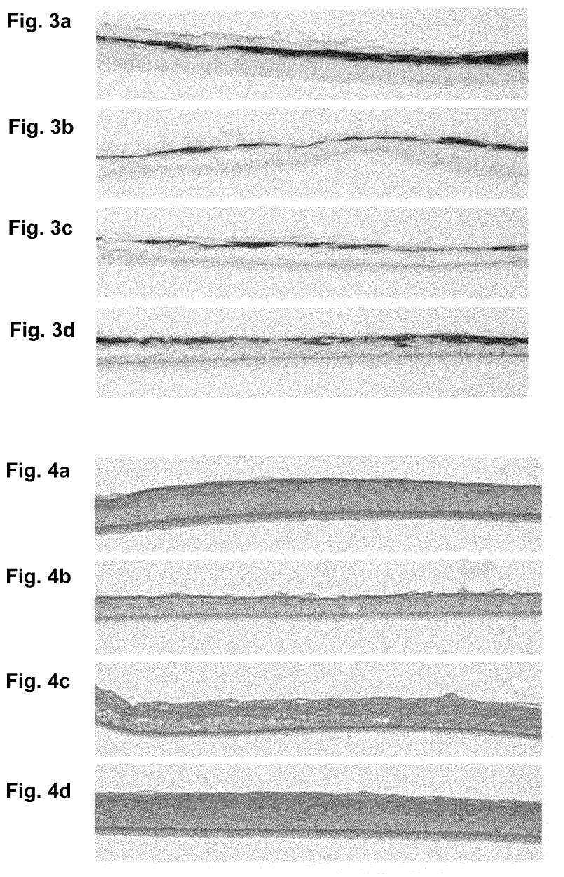

As expected, the standard condition, 1.1 mM CaCl₂·2H₂O from the basal side (condition 1), resulted in the formation of an intact 3D epidermis (FIG. 2a). The reduction of the calcium concentration to 0.3 mM during the differentiation process (condition 2) greatly impaired the formation of a dense, stratified epidermis and led to the formation of vacuoles (FIG. 2b). The treatment of the differentiating keratinocytes with 1.1 mM CaCl₂·2H₂O from the apical side (condition 3, FIG. 2c) additionally worsened the formation of a 3D epidermis. Enlarged vacuoles, which no longer ensure the integrity of the epidermis, were observed. In contrast, the apical treatment with 0.1% Ca²⁺ phospholipid complex (condition 4) improved the keratinocyte differentiation process and prevented the formation of vacuoles (FIG. 2d). Furthermore, portions of the 3D epidermis that were differentiated under the abovementioned four conditions were examined for the expression of the differentiation marker loricrin by immunohistochemical stains (cf. FIG. 3). Analogously to the normal differentiation observed in the hematoxylin-eosin staining in FIG. 2, a high level of loricrin expression was observed under normal differentiation conditions (condition 1) (FIG. 3a, dark staining of the epidermis), and a reduced level of loricrin expression was observed under reduced calcium concentrations (condition 2, FIG. 3b). The apical treatment with 0.1% Ca²⁺ phospholipid complex (condition 4) increases the expression of the epidermal differentiation marker loricrin (FIG. 3d) compared to the apical treatment with 1.1 mM CaCl₂·2H₂O (condition 3, FIG. 3c).

This means that the treatment of keratinocytes with the Ca²⁺ phospholipid complex contributes to efficient differentiation and thus correct formation of a 3D epidermis.

### Rebuilding of the Epidermis Structure in an In Vitro Skin Model, Control: Phospholipid Mixture

In a further experiment, it was investigated and checked whether the phospholipids present in the calcium phospholipid complex have an effect on 3D epidermis differentiation. Here, portions of the 3D epidermis were differentiated under the following conditions and grown for 9 days, followed by staining with hematoxylin-eosin solution:
Condition 1: 1.1 mM CaCl₂·2H₂O from the basal side (TAK-3D). As described above, this is the standard condition for efficient epidermis formation.
Condition 2: 0.3 mM CaCl₂·2H₂O from the basal side (low calcium concentration). As described above, the reduced calcium concentration causes here impaired formation of the epidermis and thus skin barrier.
Condition 3: CaCl₂·2H₂O from the basal side+0.1% phospholipid mixture (without Ca²⁺) from the apical side. Here, the phospholipid mixture was prepared as described in the section “Formation of a Ca²⁺ phospholipid complex structure”, but the dispersion was admixed with an aqueous solution without Ca²⁺, preferably at a pH in the range of 7.5-9 (prepared analogously to sample 1 in Table 1).

The apical administration, and in particular the composition of the phospholipid mixture, was carried out for comparability with condition 4.
Condition 4: 0.3 mM CaCl₂·2H₂O from the basal side+0.1% Ca²⁺ phospholipid complexes (prepared analogously to sample 6 in Table 1) from the apical side in order to imitate topical application of the phospholipid complex.

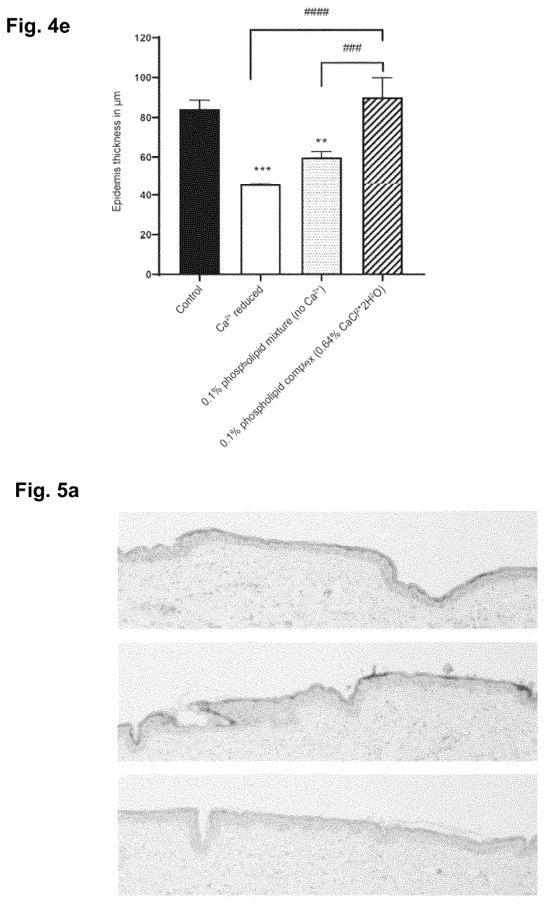

The 3D models were removed on day 9 and processed for histological analysis. The hematoxylin-eosin stains was examined for stratification thereof under a microscope, and high-resolution micrographs (10× magnification) were taken (cf. FIG. 4). The thickness of the epidermis was determined analytically on the basis of the micrographs and quantified in micrometers (μm).

As already shown in the previous experiment, the standard condition, 1.1 mM CaCl₂·2H₂O from the basal side (condition 1), resulted in the formation of an intact 3D epidermis (FIG. 4a). The quantification of epidermis thickness shows a thickness of 82.5+12.7 μm (FIG. 4e, black bar), which serves in this experiment as a reference for an intact stratified epidermis.

The reduction of the calcium concentration to 0.3 mM during the differentiation process (condition 2) again greatly impaired the formation of a dense, stratified epidermis (FIG. 4b), which is moreover characterized by a reduction in epidermis thickness to 46.0+2.5 μm (FIG. 4e, white bar).

The treatment of the differentiating keratinocytes with 0.1% phospholipid mixture without calcium from the apical side (condition 3, FIG. 4c) additionally worsened the formation of a 3D epidermis and led to the formation of vacuoles, as a result of which the integrity of the epidermis is no longer ensured and it also shows a reduced epidermis thickness of 62.2+9.8 μm (FIG. 4e, dotted bar).

In contrast, the apical treatment with 0.1% Ca²⁺ phospholipid complex (condition 4) improved the keratinocyte differentiation process and prevented the formation of vacuoles (FIG. 4d); moreover, the treatment led to an increased epidermis thickness (FIG. 4e, hatched bar).

### Influence of the Ca²⁺ Phospholipid Complex on Differentiation Marker in Skin Explants

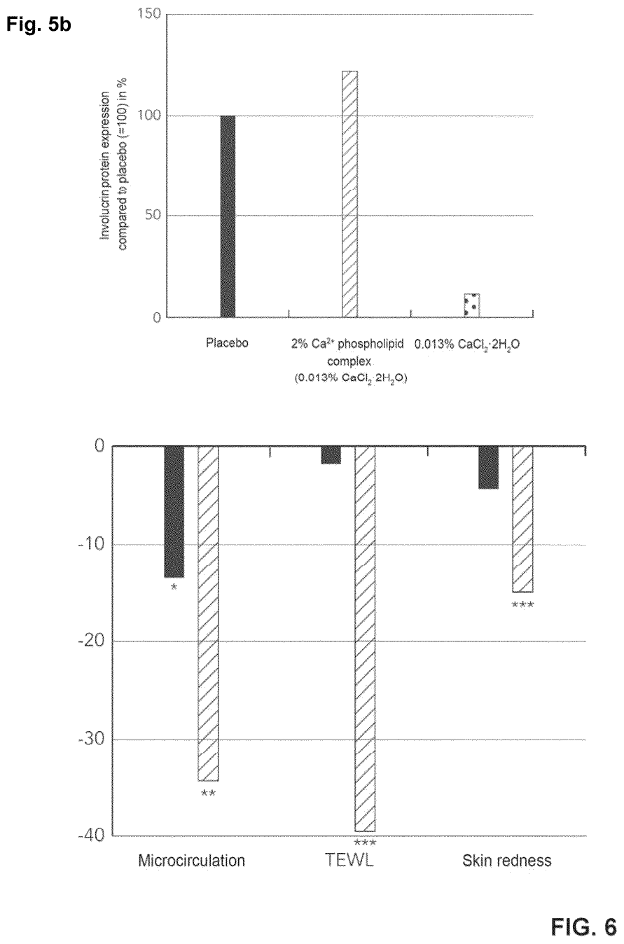

The effect of a topical treatment with Ca²⁺ phospholipid complex on the expression of the epidermis differentiation marker involucrin was investigated in skin explants. The skin explants were treated topically for 24 hours with 20 mg of the test products, based on the gel formulation from example 3 for topical application, placebo gel (without calcium), gel containing 0.013% CaCl₂·2H₂O or gel containing 2% Ca²⁺ phospholipid complex (prepared analogously to sample 6 in Table 1, corresponds to 0.013% CaCl₂·2H₂O), in Franz diffusion cells. Thereafter, the skin explants were washed with 3 ml of water and frozen at −80° C. The tissues were fixed in formaldehyde and embedded in paraffin. Tissue sections of 5 μm were prepared and mounted on glass slides before deparaffination. The slides were stained with hematoxylin-eosin stain to analyze tissue structure. The protein expression of involucrin was determined by immunohistochemical staining and quantified by image analysis. The topical treatment with 2% Ca²⁺ phospholipid complex resulted in an increase in the expression of the epidermis differentiation marker involucrin by 22.0% (FIG. 5a, middle, and 5b, hatched bar) compared to the placebo-treated skin explant (FIGS. 5a, top, and 5b, black bar). In contrast, the treatment with the corresponding concentration of CaCl₂·2H₂O resulted in a reduction of involucrin expression by 91.1% (FIG. 5a, bottom, and 5b, dotted bar) compared to the placebo-treated skin explant (FIGS. 5a, top, and 5b, black bar).

### Application of Ca²⁺ Phospholipid Complex to Stressed Skin

A randomized, placebo-controlled clinical trial investigated the Ca²⁺ phospholipid complex in respect of skin protection and regeneration efficacy. For this purpose, 20 subjects (age: 23-65 years) with normal skin were enrolled in the trial. The gel from example 3 for topical application containing 2% Ca²⁺ phospholipid complex (containing 0.64% CaCl₂·2H₂O and 5% phospholipids, analogous to sample 6 from Table 1) was used as test sample and the same gel formulation without Ca²⁺ phospholipid complex was used as placebo. The skin parameters measured were skin microcirculation (Periflux PF5000, Perimed, Sweden), transepidermal water loss (TEWL) (Tewameter®) TM300, Courage+Khazaka, Germany) and skin color (redness by means of a* parameter, Chromameter® CR-400, Minolta, Japan).

For the skin protection test, these test substances were applied twice daily for 7 days, on one forearm in each case. Furthermore, an untreated skin area on the forearm, to which no product was applied, was defined. The abovementioned skin parameters were then measured, and 2% sodium lauryl sulfate (SLS) in the form of an occlusive patch was applied to the skin for 24 h, which resulted in impairment of the skin barrier. To determine the possible protective effect of a previous treatment with 2% Ca²⁺ phospholipid complex, the skin parameters were remeasured after removal of the 2% SLS patch, and the untreated, placebo-treated area and Ca²⁺ phospholipid complex-treated area were compared. A significant reduction of all three parameters compared to the untreated and placebo-treated area was achieved by treatment with 2% Ca²⁺ phospholipid complex (FIG. 6, hatched bar). This means that pretreatment with 2% Ca²⁺ phospholipid complex protects the skin from harmful influences, thus significantly reducing the effect of stress on the skin.

Furthermore, the skin regeneration potential of the Ca²⁺ phospholipid complex was investigated in the same group of subjects. For this purpose, the abovementioned skin parameters were measured on three untreated skin areas on the forearm and a 2% SLS patch was then applied for 24 hours in each case. After removal of the SLS patch, skin parameter measurements were made every 2-3 days until day 24. The regeneration in relation to skin redness, skin microcirculation and TEWL was significantly faster in the skin region treated with 2% Ca²⁺ phospholipid complex compared to the skin region treated with the placebo gel. This confirms that the Ca²⁺ phospholipid complex has a regenerating effect on the skin and quickens normalization of the skin barrier after it has been damaged.

1. Pharmaceutical or cosmetic composition having an active content of divalent calcium (Ca²⁺) for use in topical treatment of skin with impaired barrier function, wherein the divalent calcium is present in the composition as a phospholipid complex.

2. Composition as claimed in claim 1, wherein the Ca²⁺ phospholipid complex is in the form of a complex of the Ca²⁺ with one or two phospholipids.

3. Composition as claimed in claim 1, wherein diabetes patients, are concerned.

4. Composition as claimed in claim 1, wherein the phospholipid is a phosphoglyceride selected from the following group: phosphatidic acid; phosphatidylcholine; phosphatidylethanolamine; phosphatidylinositol; phosphatidylserine; diphosphatidylglycerol, or a mixture of these systems, in hydrogenated, partially hydrogenated or nonhydrogenated form, and/or a lecithin fraction, in hydrogenated, partially hydrogenated or nonhydrogenated form.

5. Composition as claimed in claim 4, wherein the calcium phospholipid complex is a calcium complex with phospholipids, with a molar ratio of divalent calcium to phospholipids,
based on the total content of phospholipids, in the range of 0.05:1-20:1,
and/or, based on the content of negatively charged phospholipids, in the range of 0.1:1-30:1.

6. Composition as claimed in claim 4, wherein the calcium phospholipid complex is a calcium complex with negatively charged phosphatidic acid, phosphatidylinositol, and/or phosphatidylserine, with a molar ratio of divalent calcium to negatively charged phospholipids in the range of 0.2:1-1:1 or 0.3:1-0.8:1.

7. Composition as claimed in claim 1, wherein the calcium phospholipid complex is in a gel structure in the composition and/or in the starting material comprising calcium phospholipid for the composition.

8. Composition as claimed in claim 1, wherein the concentration of the divalent calcium in the Ca²⁺ phospholipid complex intended for topical application is in the range of 0.001-10 percent by weight or 0.005-5.0 percent by weight, wherein the percents by weight of CaCl₂·2H₂O are based on the entire composition,
and/or in that the concentration of the phospholipids in the Ca²⁺ phospholipid complex intended for topical application is in the range of 0.01-30 percent by weight, or in the range of 0.5-10 percent by weight, wherein the percents by weight of the phospholipids are based on the entire composition.

9. Composition as claimed in claim 1, wherein it is formulated as an ointment, cream, lotion, paste or tincture for local application to the skin of the patient.

10. Composition as claimed in claim 1, wherein it is in the form of a concentrate for preparation of a topical application formulation having a concentration of the phospholipid complex with the divalent calcium in the composition in the range of 0.1-100 percent by weight, or in the range of 1-10 percent by weight, wherein the percents by weight of the Ca²⁺ phospholipid complexes are based on the topical composition as a concentrate.

11. Method for therapeutic and/or cosmetic treatment of skin with impaired barrier function, including the skin of a diabetes patient or prediabetes patient, in claim 1 is topically applied to the skin.

12. Method as claimed in claim 11, wherein the composition as a concentrate is formulated as an ointment, cream, lotion, paste or tincture for local application to the skin of the patient.

13. Method for preparing a composition as claimed in claim 1, wherein the phospholipid starting material, optionally after prior dissolution or dispersion in an organic solvent, including ethanol, or a mixture of an organic solvent and water, is provided in an aqueous medium as a liposome structure,
and this dispersion is then admixed with an aqueous solution of Ca²⁺.

14. Method as claimed in claim 13, wherein the preparation of the liposomes and/or the homogenization is carried out with the aid of a high-pressure homogenizer.

15. Method as claimed in claim 13, wherein an ointment, cream, lotion, paste or tincture is formulated from the composition with the aid of at least one carrier material and optionally further constituents.

16. Composition as claimed in claim 1, wherein it is for use in topical treatment of the skin of diabetes patients or prediabetes patients.

17. Composition as claimed in claim 1, wherein the Ca²⁺ phospholipid complex is in the form of a complex of the Ca²⁺ with one or two phospholipids, namely with negative charges of the phospholipids, as a neutral complex structure.

18. Composition as claimed in claim 17, wherein type 2 diabetes patients are concerned.

19. Composition as claimed in claim 4, wherein the calcium phospholipid complex is a calcium complex with phospholipids, at least partially with the negatively charged phospholipids selected from the following group: phosphatidic acid, phosphatidylinositol, or phosphatidylserine, or a combination thereof, with a molar ratio of divalent calcium to phospholipids,
based on the total content of phospholipids, in the range of 0.1:1-5:1, or in the range of 0.2:1-1:1 or 0.3:1-0.8:1,
and/or, based on the content of negatively charged phospholipids, in the range of 0.3:1-10:1, or in the range of 0.4:1-5:1 or 0.5:1-1:1.

20. Composition as claimed in claim 1, wherein the concentration of the divalent calcium in the Ca²⁺ phospholipid complex intended for topical application is in the range of 0.01-0.5 percent by weight, wherein the percent by weight of CaCl₂·2H₂O is based on the entire composition.

21. Composition as claimed in claim 1, wherein it is formulated as an ointment, cream, lotion, paste or tincture for local application to the skin of the patient, wherein the composition additionally contains at least one of the following formulation constituents: thickeners, smoothing agents, moisturizers and/or humectants, surface-active agents, preservatives, antifoams, waxes, fats, oils, antioxidants and/or substances with antioxidant properties, bactericides, fungicides, perfumes, propellants, dyes, stabilizers, polar and apolar solvents, including water, pigments, UV filters, plant extracts, further active therapeutic or pharmaceutical ingredients, or a combination thereof.

22. Method according to claim 11 for therapeutic and/or cosmetic treatment of the skin of a diabetes patient or prediabetes patient, for stabilization, prevention, reduction or elimination of dry skin or moisturization of dry skin and/or for restoration of the barrier function, for stabilization, prevention, reduction or elimination of skin infections caused by damaged barrier function, by skin changes or skin irritation, including of bacterial origin, or for stabilization, prevention, reduction or elimination of fungal infections, of pigment disorders (diabetic dermopathy), of blistering, of itching (pruritus diabeticorum), of redness (including necrobiosis lipoidica diabeticorum, pseudoacanthosis nigricans, bullosis diabeticorum, rubeosis diabeticorum, scleredema diabeticorum), or of wrinkling, or of a combination of these indications, wherein a composition as claimed in claim 1 is topically applied to the skin.

23. Method according to claim 13 for preparing a composition, wherein the phospholipid starting material, optionally after prior dissolution or dispersion in an organic solvent, in particular ethanol, or a mixture of an organic solvent and water, is provided in an aqueous medium as a liposome structure, having an average particle size of less than 300 nm and or less than 200 nm,
and this dispersion is then admixed with an aqueous solution of Ca²⁺, wherein this aqueous solution has been set at a pH in the range of 7.5-10 and or in the range of 8-9, followed by homogenization to form a gel.

24. Method as claimed in claim 13, wherein the preparation of the liposomes and/or the homogenization is carried out with the aid of a high-pressure homogenizer, at a pressure of at least 500 bar or at least 1000 bar.

25. Composition as claimed in claim 1, wherein the Ca²⁺ phospholipid complex is in the form of a complex of the Ca²⁺ with one or two phospholipids, with the negative charges of the phospholipids, as a neutral complex structure, wherein the complex is in the form of a conical or double-conical, neutral structure with a central Ca²⁺ between two substantially opposite or adjacent, at least partially negatively charged phospholipids, or in the form of a neutral aggregate of such structures.
